(19) United States
(12) Patent Application Publication
(10) Pub. No.: US 2024/0148371 A1
(43) Pub. Date: May 9, 2024

(54) USER INTERFACE WITH SINGLE INPUT FOR BIOPSY DEVICE

(71) Applicant: Devicor Medical Products, Inc., Cincinnati, OH (US)

(72) Inventors: Justin REBELLINO, Cincinnati, OH (US); David C. McBREEN, Cincinnati, OH (US); Andrew P. NOCK, Dayton, OH (US); Kyle WAGNER, Mason, OH (US); Brennan Gallagher McCABE, Cincinnati, OH (US)

(21) Appl. No.: 18/400,387

(22) Filed: Dec. 29, 2023

Related U.S. Application Data
(63) Continuation-in-part of application No. 17/729,281, filed on Apr. 26, 2022.
Application No. 17/729,281 is a continuation of application No. PCT/US2020/057466, filed on Oct. 27, 2020.
(60) Provisional application No. 62/926,805, filed on Oct. 28, 2019.

(51) Int. Cl. A61B 10/02; A61B 10/00

(52) U.S. Cl. CPC A61B 10/0275; A61B 10/0041; A61B 2010/0208; A61B 2010/0225

(57) ABSTRACT

A core needle biopsy device includes a needle assembly, a drive assembly, and a user interface. The needle assembly includes a piercer and a hollow cutter. The piercer includes a sharp distal tip and a notch proximal to the distal tip. The piercer is slidably disposed within the cutter to sever a tissue sample into the notch of the piercer. The drive assembly is configured to selectively move the piercer and the cutter. The user interface includes one or more indicators and a single button. The single button is configured to initiate a plurality of movements of the drive assembly to control arming and firing of the piercer and the cutter. The one or more indicators are configured to transition between a plurality of predetermined states to define one or more predetermined status sequences indicative of movement of the piercer and the cutter via the drive assembly.

### PRIORITY

This application is a continuation-in-part of U.S. Ser. No. 17/729,281, entitled “User Interface for Biopsy Device,” filed on Apr. 26, 2022, which is a continuation of International Application No. PCT/US2020/057466, filed on Oct. 27, 2020, which claims priority to U.S. Provisional Application Ser. No. 62/926,805, entitled “User Interface for Biopsy Device,” filed on Oct. 28, 2019, the disclosures of which are incorporated by reference herein.

### BACKGROUND

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

One technique for collecting a breast biopsy is to use a core needle biopsy device. Core needle biopsy devices may use a sharp, solid piercer equipped with a lateral tissue receiving notch positioned adjacent to the distal end of the piercer. When tissue is received within the notch, an elongate hollow cutting sheath is translated over the notch to sever a tissue sample. The severed tissue sample is then stored within the notch until both the piercer and the cutting sheath are removed from the patient. Thus, in core-needle biopsy devices, only one tissue sample can be collected per insertion of the piercer and cutting sheath.

Another technique for conducting a breast biopsy is to conduct a breast biopsy using a vacuum-assisted breast biopsy device. In contrast to core needle breast biopsy procedures, vacuum-assisted breast biopsy devices permit the probe to remove multiple samples without requiring the probe be removed from the breast after every sample is collected. For instance, in a vacuum assisted breast biopsy device, a hollow needle is used to penetrate tissue. The hollow needle includes a lateral aperture adjacent to a sharp distal tip. A hollow cutter is disposed within the hollow needle and is moved axially relative to the lateral aperture of the needle to sever tissue samples. Once a tissue sample is severed by the hollow cutter, the tissue sample is transported axially though the cutter and collected in a tissue collection feature.

Examples of vacuum assisted biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,938,786, entitled “Vacuum Timing Algorithm for Biopsy Device,” issued May 10, 2011; U.S. Pat. No. 8,118,755, entitled “Biopsy Sample Storage,” issued Feb. 1, 2012; and U.S. Pat. No. 8,206,316, entitled “Tetherless Biopsy Device with Reusable Portion,” issued on Jun. 26, 2012. The disclosure of each of the above-cited U.S. patents is incorporated by reference herein.

Examples of core needle biopsy devices are disclosed in U.S. Pat. No. 5,560,373, entitled “Needle Core Biopsy Instrument with Durable or Disposable Cannula Assembly,” issued on Oct. 1, 1996; U.S. Pat. No. 5,817,033, entitled “Needle Core Biopsy Device,” issued on Oct. 6, 1998; and U.S. Pat. No. 5,511,556, entitled “Needle Core Biopsy Instrument,” issued on Apr. 30, 1996. The disclosure of each of the above-cited U.S. patents is incorporated by reference herein.

A challenge in both biopsy device configurations described above is operating the biopsy device with real-time feedback and limited user interaction points. Such challenges can arise due to the unique piercer and cutter configuration that is encountered in the context of core needle biopsy devices. For instance, due to the relatively simple nature of core needle biopsy devices, the piercer and/or cutting sheath can be driven by a spring actuated mechanism. However, such mechanisms can require multiple buttons or user input features to engage different movements during a tissue acquisition sequence. The multiplicity of user input features can be undesirable in some examples due to operator confusion. In addition, such mechanisms provide limited means for operator feedback, thereby compounding operator confusion. Thus, certain user interface features may be desirable for integration into a biopsy device that simplify the operator experience while also providing real-time feedback.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary core needle biopsy device;

FIG. 2 depicts an exploded view of a needle assembly of the core needle biopsy device of FIG. 1;

FIG. 3 depicts a perspective view of the distal portion of the needle assembly of FIG. 2;

FIG. 4 depicts a perspective view of a drive assembly of the core needle biopsy device of FIG. 1;

FIG. 5 depicts a perspective view of a tissue sample holder of the core needle biopsy device of FIG. 1;

FIG. 6 depicts a detailed perspective view of an exemplary user interface of the core needle biopsy device of FIG. 1;

FIG. 7 depicts a schematic flow chart of a use of the core needle biopsy device of FIG. 1, with various states of the user interface of FIG. 6;

FIG. 8 depicts a detailed top plan view of the user interface of FIG. 6, in a first state;

FIG. 9 depicts another detailed top plan view of the user interface of FIG. 6, in a second state;

FIG. 10 depicts still another detailed top plan view of the user interface of FIG. 6, in a third state;

FIG. 11 depicts yet another detailed top plan view of the user interface of FIG. 6, in a fourth state;

FIG. 12 depicts yet another detailed top plan view of the user interface of FIG. 6, in a fifth state;

FIG. 13 depicts yet another detailed top plan view of the user interface of FIG. 6, in a sixth state;

FIG. 14 depicts yet another detailed top plan view of the user interface of FIG. 6, in a seventh state;

FIG. 15 depicts yet another detailed top plan view of the user interface of FIG. 6, in an eighth state;

FIG. 16 depicts yet another detailed top plan view of the user interface of FIG. 6, in a ninth state; and

FIG. 17 depicts yet another detailed top plan view of the user interface of FIG. 6, in a tenth state.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Biopsy devices may be used to collect tissue samples in a variety of ways. For example, in some instances tissue samples are collected into a single tissue basket such that all tissue samples collected during a given biopsy procedure are deposited into the single tissue sample basket. In some other instances, tissue samples are collected into a tissue sample holder having separate compartments for each collected tissue sample. Such a multi-compartment tissue sample holder may additionally include trays or strips that individually hold each tissue sample separately from the other tissue samples. Such trays or strips may be removable or otherwise separable from the tissue sample holder at the conclusion of a biopsy procedure.

Regardless of the structure in which the tissue samples are stored, tissue samples may be collected using biopsy devices under the guidance of various imaging modalities such as ultrasound image guidance, stereotactic (X-ray) guidance, MRI guidance, Positron Emission Mammography (“PEM” guidance), Breast-Specific Gamma Imaging (“BSGI”) guidance, or otherwise. Each procedure has its own methodology based on the form of imaging guidance used.

Vacuum assisted biopsy devices and core needle biopsy devices both may have various advantages over the other, depending on context. For instance, one advantage of vacuum assisted biopsy devices is that vacuum assistance permits removal of multiple tissue samples using a single insertion. However, while core needle biopsy devices lack this feature, use of core needle biopsy devices may still be desirable. For instance, core needle biopsy devices are generally capable of having smaller needles relative to core needle biopsy devices, thereby reducing patient anxiety and increasing the capacity of the needle to penetrate a lesion. Therefore, in some instances it may be desirable to incorporate the feature of multiple sample removal of a vacuum assisted biopsy device into a core needle biopsy device to achieve the benefits present in both styles of biopsy device.

A desirable feature of the device described herein, which is a core needle biopsy device, is that the device allows for single insertion with multiple samples being obtained whilst using a core needle type device. To facilitate this functionality, the biopsy device further includes a tissue sample holder having one or more features to facilitate collection of a severed tissue sample from a notch, dugout, aperture, and/or other sample collection feature.

### I. EXEMPLARY CORE NEEDLE BIOPSY DEVICE WITH MULTI-SAMPLE COLLECTION

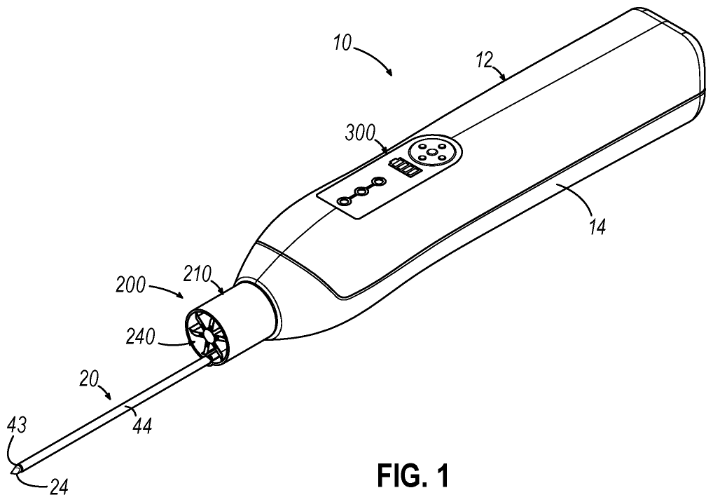

FIG. 1 shows an exemplary core needle biopsy device (10) for use in a breast biopsy procedure. Core needle biopsy device (10) of the present example comprises a body (12) and a needle assembly (20) extending distally from body (12). Body (12) includes an outer housing (14) and a user interface (300) disposed on outer housing (14). As will be described in greater detail below, outer housing (14) encloses various components of biopsy device (10). As will also be described in greater detail below, such components can be activated or otherwise controlled by user interface (300) to drive needle assembly (20) through a cutting cycle and a tissue acquisition cycle. To this end, outer housing (14) of the present example is sized and shaped for grasping by an operator using a single hand. Similarly, user interface (300) is positioned for actuation by a single hand. Although not shown, it should be understood that in some examples outer housing (14) may comprise multiple parts such that each part interconnects to form outer housing (14). For instance, in some examples outer housing (14) can be formed by the combination of a disposable probe and a reusable holster.

A. Exemplary Needle Assembly

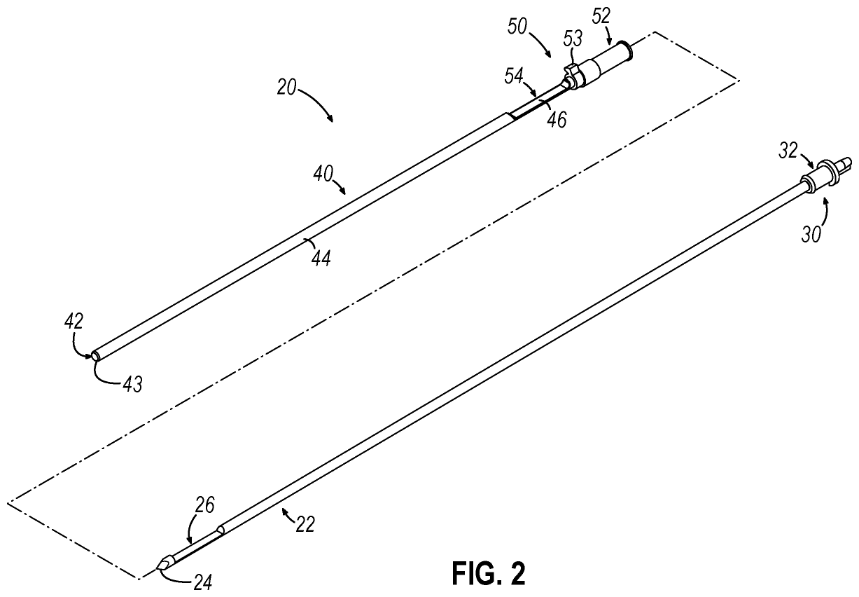

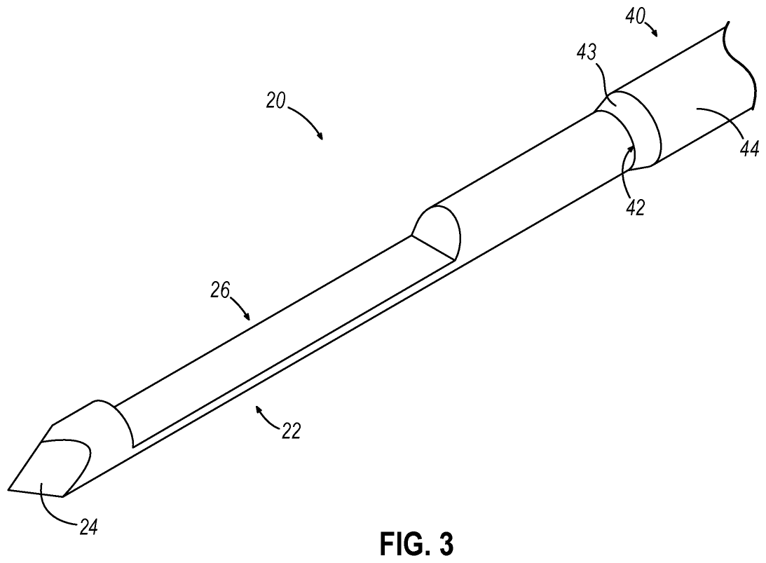

FIGS. 2 and 3 show needle assembly (20) in greater detail. As can be seen in FIG. 2, needle assembly (20) comprises an elongate piercer (22) and an elongate cutter (40). As will be described in greater detail below, piercer (22) is generally movable relative to cutter (40) to pierce tissue and collect tissue samples, while cutter (40) is generally movable relative to piercer (22) to sever tissue samples. Piercer (22) comprises a generally cylindrical rod having a sharp distal tip (24) and a notch (26) disposed adjacent to distal tip (24). As will be described in greater detail below, distal tip (24) is generally configured to penetrate tissue of a patient. As will also be described in greater detail below, notch (26) is generally configured to receive tissue therein such that a tissue sample may be collected within notch (26) after the tissue sample is severed by cutter (40).

An end portion (30) is disposed on the proximal end of piercer (22). End portion (30) of the present example is overmolded onto the proximal end of piercer (22) and is generally configured to enhance the manipulability of piercer (22). In particular, end portion (30) comprises a receiving feature (32) in the form of a cylindrical indentation or notch. Receiving feature (32) is configured to receive a portion of a piercer drive assembly (300). As will be described in greater detail below, this permits piercer drive assembly (300) to drive movement of piercer (22) through a predetermined sequence of movement.

Cutter (40) comprises a generally hollow cylindrical tube that is configured to receive piercer (22) therein. Cutter (40) comprises an open distal end (42), a cannula portion (44) and an end portion (50). Open distal end (42) is configured to permit at least a portion of piercer (22) to protrude from cutter (40) when piercer (22) is moved relative to cutter (40). As will be described in greater detail below, this configuration permits needle assembly (20) to move through the cutting cycle and the tissue acquisition cycle by permitting notch (26) of piercer (22) to move relative to distal end (42) of cutter (40).

Open distal end (42) of the present example includes a tapered edge (43). Tapered edge (43) is generally configured to slice through tissue to separate tissue samples when cutter (40) is moved relative to notch (26) of piercer (22). Thus, it should be understood that tapered edge (43) is generally configured to act as a blade. Although the present example is described and shown as using a tapered configuration, it should be understood that in other examples various alternative configurations can be used. For instance, in some examples tapered edge (43) includes a plurality of serrations in addition or in alternative to the taper shown. In still other examples, tapered edge (43) can include any other additional or alternative cutting surface as will be apparent to those of ordinary skill in the art in view of the teachings herein.

Cannula portion (44) of cutter (40) extends proximally from distal end (42) through end portion (50) such that piercer (22) can be received with the proximal end of cutter (40). Unlike end portion (30) of piercer (22), end portion (50) of cutter (40) is generally elongate such that end portion (50) can accommodate additional features that will be described in greater detail below. In the present example, the distal extension of end portion (50) can be relative to outer housing (14) to permit a portion of end portion (50) to be accessible to an operator for tissue sample collection purposes. A merely illustrative example of a tissue collection mechanism associated with end portion (50) will be described in greater detail below.

End portion (50) of cutter (40) comprises a receiving feature (52) and a tissue collection feature (54). As with receiving feature (32) of piercer (22), receiving feature (52) of end portion (50) comprises a cylindrical indentation, slot, or other receiving feature that is configured to receive at least a portion of a cutter drive assembly (200). As will be described in greater detail below, receiving feature (52) is configured to receive at least a portion of cutter drive assembly (200) to permit cutter drive assembly (200) to move cutter (40) through a predetermined sequence of movement.

Tissue collection feature (54) is disposed distally relative to receiving feature (52). Tissue collection feature (54) generally defines an elongate notch that is open to cannula portion (44) of cutter (40). Thus, cannula portion (44) includes a cutout portion (46) that is adjacent to, or otherwise defines, tissue collection feature (54). Accordingly, it should be understood that tissue collection feature (54) is in communication with the hollow interior, or a lumen, defined by cannula portion (44). As will be described in greater detail below, this relationship between tissue collection feature (54) and cannula portion (44) permits an operator to remove tissue samples from cutter (40) as they are collected by piercer (22).

End portion (50) further includes a driver (53) extending outwardly from an outer surface of end portion (50). Driver (53) generally comprises a square or rectangular shape. As will be described in greater detail below, driver (53) is generally configured to manipulate certain features associated with various tissue collection features described herein. Although driver (53) of the present example is shown in connection with end portion (50), it should be understood that in other examples driver (53) can be associated with other components or omitted entirely.

FIG. 3 shows piercer (22) disposed within cutter (40). As can be seen, cutter (40) is generally configured to receive piercer (22) such that piercer (22) is coaxial with cutter (40). In addition, piercer (22) is generally movable relative to open distal end (42) of cutter (40). It should be understood that in some circumstances piercer (22) moves relative to cutter (40), while cutter (40) remains stationary. In other circumstances, cutter (40) moves relative to piercer (22), while piercer (22) remains stationary. In either case, it should be understood that piercer (22) and cutter (40) are generally configured such that notch (26) of piercer (22) moves into and out of cutter (40) such that notch (26) can be disposed distally or proximally relative to open distal end (42) of cutter (40). As will be described in greater detail below, this configuration permits piercer (22) and cutter (40) to cooperate to pierce tissue, cut a tissue sample, and retract the tissue sample for collection by an operator via tissue collection feature (54).

B. Exemplary Drive Assembly

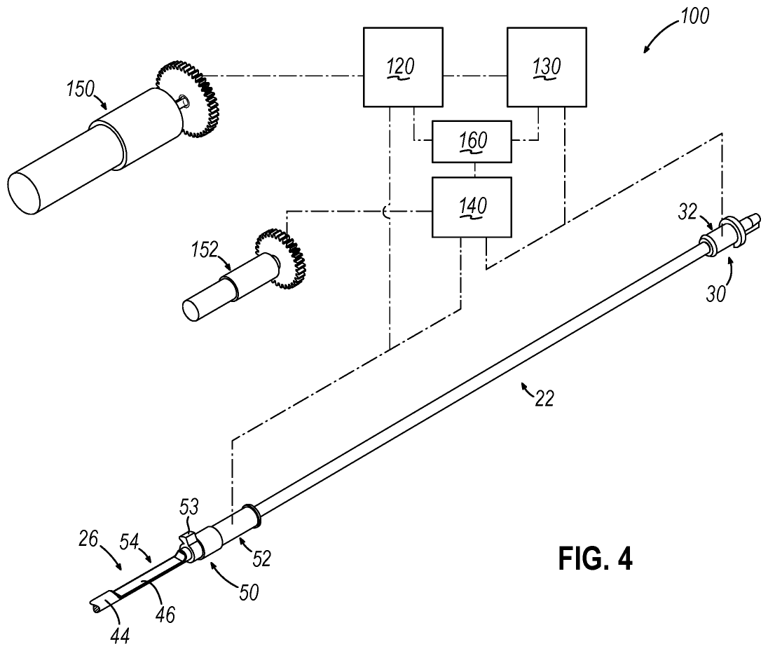

FIG. 4 shows the internal components of body (12) of biopsy device (10) with outer housing (14) removed. As can be seen, inside outer housing (14), body (12) includes a drive assembly (100). Drive assembly (100) is generally configured to engage needle assembly (20) to drive piercer (22) and cutter (40) through a predetermined sequence of movements to thereby pierce tissue and acquire a plurality of tissue samples with a single insertion of needle assembly (20) into a patient. Although not shown, it should be understood that outer housing (14) defines various internal geometries that support or otherwise engage drive assembly (100). As will be understood, such internal geometries are used to provide relative movement of various components of drive assembly (100) relative to other components of drive assembly (100) and/or outer housing (14).

Drive assembly (100) comprises a cutter drive assembly (120), a piercer drive assembly (130), and a firing assembly (140). Generally, firing assembly (140) is configured to cock (or arm) and fire cutter (40) and piercer (22) in a predetermined sequence to sever a tissue sample. To collect the severed tissue sample, cutter drive assembly (120) is generally configured to retract cutter (40). Similarly, piercer drive assembly (130) is generally configured to retract piercer (22). It should be understood that, in some examples, both cutter drive assembly (120) and piercer drive assembly (130) can be configured to rotate cutter (40) and/or piercer (22), respectively.

Firing assembly (140) is generally shown schematically in the present example. Thus, it should be understood that in some examples firing assembly (140) can take on a variety of forms having a combination of gears, racks, leadscrews, carriages, springs, and/or etc. Such components of firing assembly (140) can generally be configured to rapidly fire cutter (40) and piercer (22) in a predetermined sequence to penetrate tissue. For instance, in some examples, firing assembly (140) is configured to rapidly fire piercer (22) distally to penetrate tissue. Firing assembly (140) is also configured to rapidly fire cutter (40) distally. The firing of cutter (40) can be either delayed relative to piercer (22) or slower relative to piercer (22) such that notch (26) can be exposed relative to cutter (40). This sequence can permit tissue to enter notch (26), so that it can be severed by subsequent movement of cutter (40). In addition, it should be understood that firing assembly (140) can include other components and/or features to permit cocking of cutter (40) and/or piercer (22) prior to firing.

Cutter drive assembly (120) is generally configured to translate and/or rotate cutter (40) either independently of piercer (22) or in concert therewith. For instance, cutter drive assembly (120) can include various combinations of gears, racks, leadscrews, carriages, springs, and/or etc. to drive cutter (40) through a predetermined sequence. In one such sequence, cutter (40) is retracted proximally relative to outer housing (14) to prepare cutter (40) for a tissue collection sequence that will be described in additional detail below. In addition, cutter drive assembly (120) can also be configured to rotate cutter (40) in a predetermined sequence to assist with the tissue collection sequence described in greater detail below.

Piercer drive assembly (130) is generally configured to translate and/or rotate piercer (22) either independently of cutter (40) or in concert therewith. For instance, piercer drive assembly (120) can include various combinations of gears, racks, leadscrews, carriages, springs, and/or etc. to drive piercer (22) through a predetermined sequence. In one such sequence, piercer (22) is retracted proximally relative to cutter (40) after severing a tissue sample to retract the tissue sample proximally towards outer housing (14). Once piercer (22) is retracted, the tissue sample can be extracted for collection in the tissue collection sequence described in greater detail below.

In the present example, drive assembly (100) is powered by one or more motors (150, 152). In particular, drive assembly (100) of the present example includes a drive motor (150) and a firing motor (152). Drive motor (150) of the present example is in communication with both cutter drive assembly (120) and piercer drive assembly (130) to provide rotational motion to both assemblies, which ultimately drives translation and/or rotation of both cutter (40) and piercer (22). Similarly, firing motor (152) is in communication with firing assembly (140) to drive firing and/or cocking of cutter (40) and piercer (22). Although drive assembly (100) of the present example includes two motors (150, 152), it should be understood that in other examples any suitable number of motors may be used such as a single motor, or three or more motors. In addition, motors (150, 152) can be configured to drive cutter drive assembly (120), piercer drive assembly (130), and/or firing assembly (140) in various combinations.

Although cutter drive assembly (120), piercer drive assembly (130), and firing assembly (140) of the present example are shown schematically as three separate drive assemblies, it should be understood that in other examples various elements of cutter drive assembly (120), piercer drive assembly (130), and firing assembly (140) can be combined into a single drive assembly or multiple drive assemblies to drive motion of cutter (40) and piercer (22) in accordance with the sequences described herein. In some examples, cutter drive assembly (120), piercer drive assembly (130), and firing assembly (140) can be constructed in accordance with at least some of the teachings of U.S. Ser. No. 16/381,573, entitled “Core Needle Biopsy Device for Collecting Multiple Samples in a Single Insertion,” filed on Apr. 11, 2019, the disclosure of which is incorporated by reference herein.

C. Exemplary Tissue Sample Holder

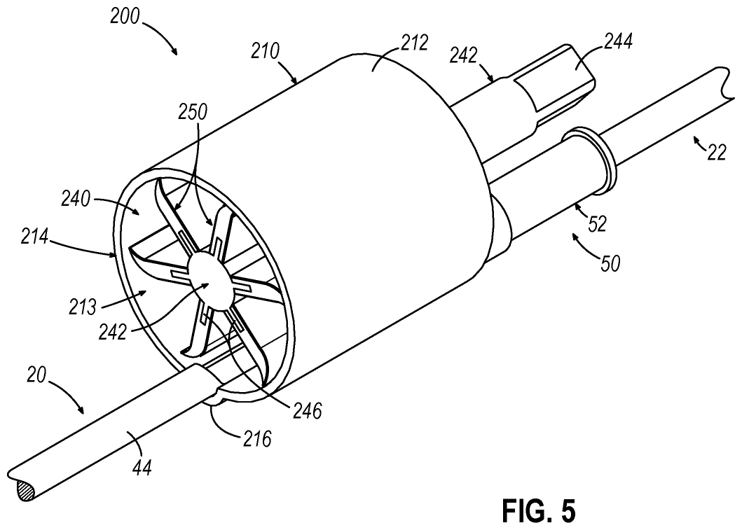

As shown in FIG. 5, biopsy device (10) of the present example includes a tissue sample holder (200) for the collection of multiple samples during a single insertion of needle assembly (20). Tissue sample holder (200) of the present example includes an extraction mechanism (240) disposed within a cylindrical outer housing (210). Tissue sample holder (200) is generally configured to collect a plurality of tissue samples from tissue collection feature (54) of needle assembly (20) during a biopsy procedure using rotation of extraction mechanism (240). As will be described in greater detail below, tissue sample holder (200) is generally configured to collect and store six tissue samples, although any suitable number can be collected and stored in other examples.

Outer housing (210) includes a cylindrical body (212) defining a sample chamber (213) and an open distal end (214), a closed proximal end (not shown), and a needle receiving portion (216) extending between open distal end (214) and the closed proximal end. In the present example, outer housing (210) is generally transparent to promote visibility of tissue samples during sample collection. Although outer housing (210) of the present example is shown as having open distal end (214), it should be understood that in other examples, open distal end (214) can be closed or capped to seal sample chamber (213) of outer housing (210) relative to the environment.

Needle receiving portion (216) is generally configured as a semi-cylindrical indentation or bulge in the otherwise cylindrical shape of outer housing (210). Needle receiving portion (216) is generally sized to correspond to the size and shape of needle assembly (20). Thus, needle receiving portion (216) generally defines a pocket or recessed area where needle assembly (20) can rest.

Extraction mechanism (240) includes a shaft (242) and a plurality of wipers (250) arranged around shaft (242). Shaft (242) is generally rotatable to thereby rotate wipers (250) within outer housing (210) to collect and store tissue samples as each tissue sample is collected by needle assembly (20). The proximal end of shaft (242) includes a keyed portion (244) that is configured to communicate with either a manual or motorized driver to rotate shaft (242).

The distal end of shaft (242) includes a plurality of couplers (246) extending outwardly from an exterior surface of shaft (242). Each coupler (246) is generally configured to receive a corresponding wiper (250) to provide a secure base for each wiper (250) to fasten to. Each coupler (246) of the present example defines a generally rectangular cross-section. In other examples, various alternative cross-sectional shapes can be used such as triangular, circular, square, or the like. Although not shown, it should be understood that couplers (246) can extend axially along the length of shaft (242) for a length approximately equivalent to the length of each wiper (250).

Each wiper (250) defines a generally curved or wave-shaped surface on the outer end. In the present curved shape, there is a concavity that is oriented in the direction of rotation of shaft (242). The particular shape of each wiper (250) is generally configured to atraumatically engage a tissue sample to manipulate the tissue sample out of tissue collection feature (54) and into sample chamber (213) of outer housing (210). Although each wiper (250) of the present example has a curved shape, it should be understood that in other examples various other shapes can be used such as rounded, square, triangular, and/or etc. In addition, although each wiper (250) is shown as having a generally consistent shape longitudinally, it should be understood that in some examples, the shape can be varied as wiper (250) extends axially.

Wipers (250) are generally formed of a flexible yet partially resilient material such as rubber or elastomer. For instance, wipers (250) are generally flexible enough to flex around the interface between outer housing (210) and needle assembly (20). This flexibility can be generally desirable to reduce trauma when each wiper (250) engages tissue, while also promoting complete engagement between each wiper (250) and tissue. Meanwhile, at least some resiliency is provided so that each wiper (250) can push or otherwise move a tissue sample. In some examples, the flexibility of each wiper (250) can be characterized in terms of a durometer. Although several suitable durometers can be used, one suitable durometer range is 30 to 80.

Each coupler (246) and wiper (250) is generally arranged around shaft (242) in an angularly spaced manner such that wipers (250) are spaced equal angular distances from each other. This generally results in couplers (246) and wipers (250) collectively forming a starburst pattern. This configuration may be desirable to divide sample chamber (213) into six equal segments for the storage of tissue samples. However, it should be understood that in other examples, other suitable spacing can be used including unequal spacing.

Although not shown, it should be understood that tissue sample holder (200) can have multiple different configurations for the collection of one or more tissue samples. By way of example only, in some examples, tissue sample holster (200) can be configured in accordance with any one or more of the teachings of U.S. Ser. No. 62/916,277, entitled “Sample Management for Core Needle Biopsy Device,” filed on Oct. 17, 2019, the disclosure of which is incorporated by reference herein.

In use, collection of a tissue sample using tissue sample holder (200) can begin after cutter (40) and piercer (22) have been driven by drive assembly (100) to sever and collect a tissue sample. In particular, once the tissue sample has been severed, the tissue sample is transported to tissue collection feature (54) using notch (26) of piercer (22).

In the present example, tissue sample holder (200) is positioned along the axis of needle assembly (20) such that each wiper (250) is aligned with tissue collection feature (54). Accordingly, to collect the tissue sample, shaft (242) can be rotated to rotate each wiper (250) within sample chamber (213) to sweep a selected wiper (250) adjacent to tissue collection feature (54) across notch (26). As the selected wiper (250) sweeps across notch (26), the selected wiper (250) engages the tissue sample to push the tissue sample out of tissue collection feature (54).

Once the selected wiper (250) sweeps across notch (26), rotation of shaft can continue. Continued rotation results in the tissue sample being moved around the interior of outer housing (210) to permit the tissue sample to be stored and ready needle assembly (20) for collection of further tissue samples. At this stage, rotation of shaft (242) can continue in coordination with sequential movement of cutter (40) and piercer (22) for the severing and collection of another tissue sample. Alternatively, rotation of shaft (242) can temporarily cease to permit cutter (40) and piercer (22) to reposition and collect another tissue sample. Regardless, once another tissue sample is collected, rotation of shaft (242) can be used to sweep another wiper (250) across notch (26) to collect another tissue sample. The same process can then be repeated any suitable number of times until tissue sample holder (200) is full or a desired number of tissue samples have been collected.

### II. EXEMPLARY USER INTERFACE

In some examples, it may be desirable to incorporate certain user interface features into biopsy device (10) that provide an operator with real-time feedback and simplified interaction. For instance, as described above, drive assembly (100) and firing assembly (140) are configured to drive needle assembly (20) in a predetermined sequence of movements to collect one or more tissue samples. In some examples, this sequence of movements can be controlled at various points during the sequence. However, such control can may lead to the addition of multiple buttons or other operator input features. In some examples, this configuration may be undesirable because multiple operator input features could lead to operator confusion. Thus, it may be desirable to limit the particular number of operator input features, while still providing some control. In addition, the predetermined sequence described above can also lead to operator confusion due to limited operator feedback during the sequence of movements. Thus, it may be desirable in some examples to include various indicators to provide real-time feedback related to the status of biopsy device (10). Although an exemplary user interface (300) is described below, it should be understood that various alternative user interfaces may be used without departing from the teachings included herein.

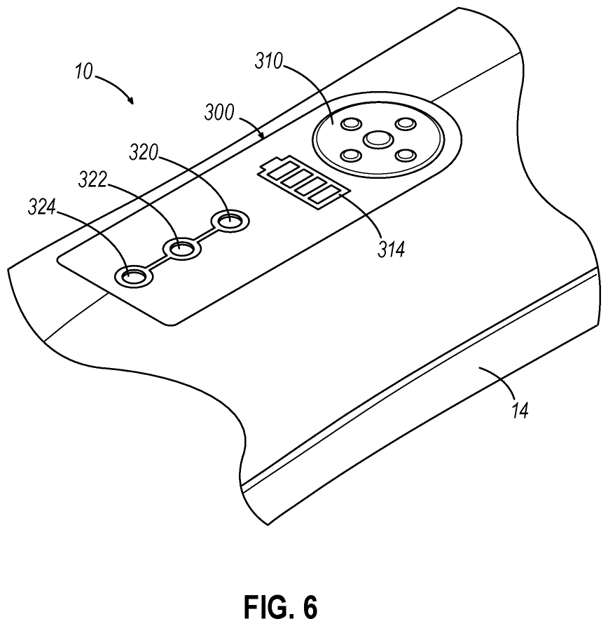

FIG. 6 shows an exemplary user interface (300) that can be readily incorporated into biopsy device (10) described above. User interface (300) of the present example is generally configured to control operation of drive assembly (100) and firing assembly (140) to collect one or more tissue samples using needle assembly (20). In addition, user interface (300) is generally configured to provide real-time feedback to an operator as to the status of biopsy device (10) as drive assembly (100) and firing assembly (140) move needle assembly (20) through various operational states.

User interface (300) includes a button or actuator (310) (also referred to as a single button), a power indicator (314), and a series of indicators (320, 322, 324) related to the operational status of biopsy device (10). Actuator (310) of the present example is in communication with drive assembly (100) and/or firing assembly (140) either by direct electrical communication, direct mechanical communication, or some combination thereof. Alternatively, in some examples, actuator (310) is in communication with a controller or other electronic circuitry configured to communicate with drive assembly (100) and/or firing assembly (140). In such examples, controller includes a processor and memory, with the processor being configured to implement one or more instructions stored in the memory. The controller may then be in communication with one or more aspects of drive assembly (100) and/or firing assembly (140) (e.g., one or more motors) to control drive assembly (100) and/or firing assembly (140) using input from actuator (310). Regardless, it should be understood that actuator (310) is generally configured to control the operation of drive assembly (100) and/or firing assembly (140). As will be described in greater detail below, this generally can involve an operator pressing or otherwise manipulating actuator (310) to cause drive assembly (100) and/or firing assembly (140) to move needle assembly (20) through various predetermined operational sequences.

Actuator (310) of the present example is configured in a single button configuration. As will be described in greater detail below, actuator (310) can be pressed at various stages to initiate one or more movements of drive assembly (100) and/or firing assembly (140). As will also be described in greater detail below, use of actuator (310) can include single discrete actuations or movements. In addition, or in the alternative, use of actuator (310) can include more complex movement sequences such as holds (also referred to as long presses), double presses, triple presses, and/or etc. In some examples, such more complex movement sequences of actuator (310) may be desirable to provide more complex control features to drive assembly (100) and/or firing assembly (140).

Actuator (310) of the present example is positioned relative to outer housing (14) of body (12) for manipulation by a single hand when outer housing (14) is grasped by such a single hand. In particular, actuator (310) is generally positioned distally along a longitudinal axis defined by outer housing (14). In this distal position, actuator (310) is proximate the distal end of outer housing (14) and oriented proximally of tissue sample holder (200). Additionally, actuator (310) is positioned proximally relative to one or more other features of user interface (300). The proximal position of actuator (310) relative to other components of user interface (300) may be desirable to prevent such other components of user interface (300) from being obscured by one or more fingers being used to engage actuator (310). This longitudinal positioning may be desirable to provide a region of outer housing (14) proximal of actuator (310) for gasping by a single hand. Additionally, actuator (310) is transversely centered with respect to outer housing (14). Such transverse centering may be desirable to permit actuation of actuator (310) by the left or right hand of a user, thereby facilitating left-handed or right-handed use.

Power indicator (314) is positioned distally of actuator (310) and is generally configured to provide an indication of the power status of biopsy device (10). In particular, power indicator (314) of the present example comprises a row of a plurality of light emitting diodes (LED) configured illuminate in a predetermined sequence. Biopsy device (10) of the present example is battery (160) operated. Thus, the sequential illumination of the LEDs of power indicator (314) is configured to provide an indication of the power remaining within battery (160). Although power indicator (314) of the present example includes four LED's, it should be understood that in other examples any suitable number of LED's can be used.

The series of indicators (320, 322, 324) includes a proximal indicator (320), an intermediate indicator (322), and a distal indicator (324). Generally, each indicator (320, 322, 324) is configured to independently transition through a plurality of states to communicate certain status information related to biopsy device (10). For instance, in the present example, each indicator (320, 322, 324) is an LED configured to transition between an off state (not emitting light), a solid on state (emitting light), and a blinking on state (periodically emitting light). However, it should be understood that in other examples the particular states used can be varied. For instance, in some examples, indicators (320, 322, 324) are configured to emit a variety of different colors and such colors can be used to communicate certain status information related to biopsy device (10). In other examples, illumination of indicators (320, 322, 324) can be controlled to blink each indicator (320, 322, 324) at a variable rate to communicate certain status information related to biopsy device (10). Of course, various alterative states may be used as will be apparent to those of ordinary skill in the art in view of the teachings herein.

As noted above, indicators (320, 322, 324) are configured as three separate LEDs. However, it should be understood should be understood that in other examples various alternative configurations can be used. For instance, in some examples, indicators (320, 322, 324) can be formed by a single LED screen graphically divided into a plurality of segments. Such segments can then be illuminated similar to the illumination described above. Alternatively, the illumination described above can be replaced with symbols, graphics, numbers, emojis, and/or etc. to provide the same status information through alternative means.

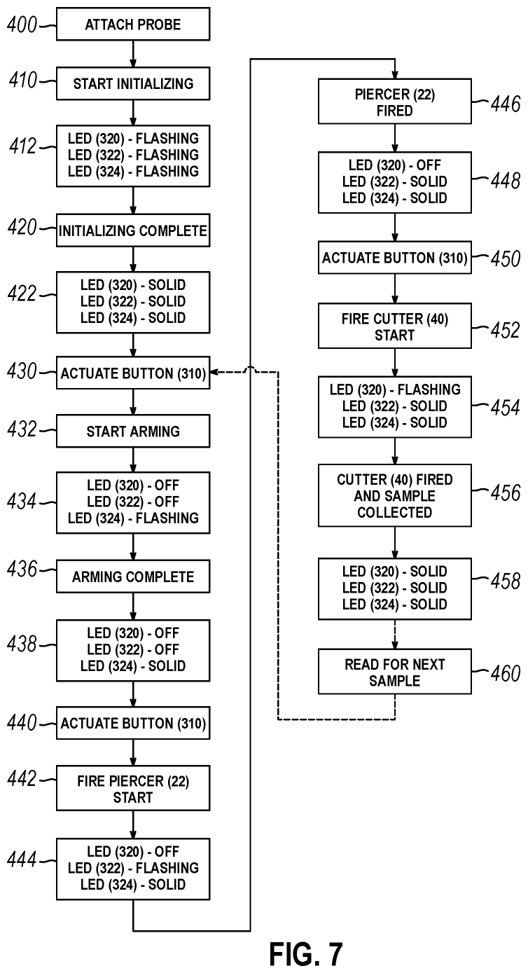

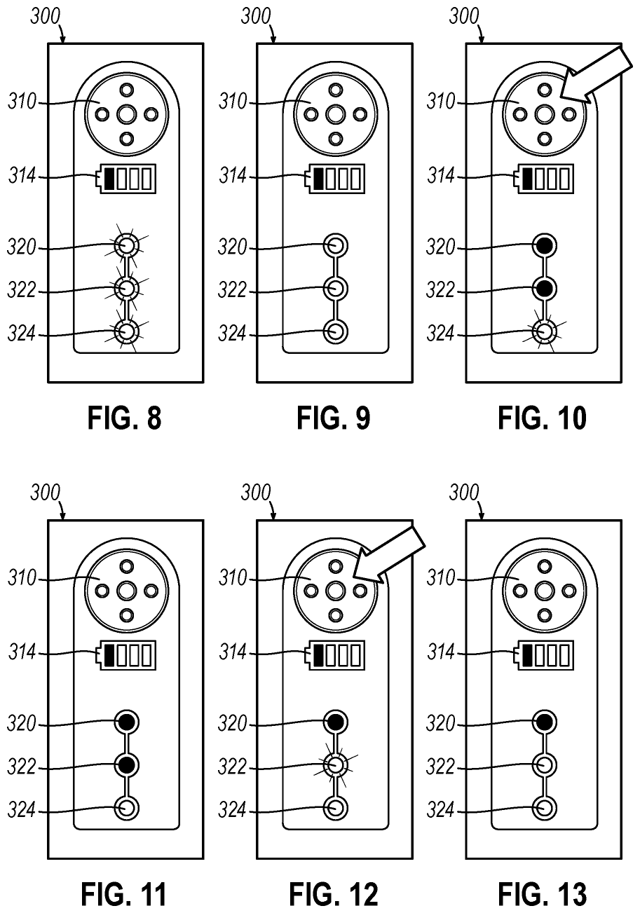

FIGS. 7-15 show an exemplary sequence of operation that can be used to communicate the operational status of biopsy device (10) to an operator via indicators (320, 322, 324). In an initial state, outer housing (14) is divided such that a probe is separated from holster. To begin the sequence the probe is attached to the holster as indicated in block (400) to completely form outer housing (14). Once probe is attached to holster as indicated in block (400), an initialization sequence begins automatically as indicated by block (410). As biopsy device (10) progresses through the initialization sequence, proximal indicator (320), intermediate indicator (322), and distal indicator (324) all flash or blink in unison as shown in block (412) to communicate that biopsy device (10) is progressing through the initialization sequence. The status of each indicator (320, 322, 324) as shown in block (412) is shown graphically in FIG. 8. Although initialization begins automatically in the present use by attachment of probe to holster, in other uses, initialization can be initiated using actuator (310). For instance, actuator (310) can be pressed to initiate initialization. Alternatively, in other uses, actuator (310) can be pressed in a predetermined sequence to initiate initialization. Such a predetermined sequence may include, for example, a 2 or more second hold (also referred to as a long press), a double tap, a triple tap, or a combination thereof. Such predetermined sequences may be desirable in some uses to avoid inadvertent initiation of initialization.

Once initialization has completed, biopsy device (10) stops automatically. Meanwhile, proximal indicator (320), intermediate indicator (322), and distal indicator (324) all switch from flashing or blinking to continuously illuminating light or otherwise being solidly highlighted. This state of indicators (320, 322, 324) is shown schematically in block (422) of FIG. 7 and graphically in FIG. 9.

When indicators are all continuously illuminated (320, 322, 324) as shown schematically in block (422) of FIG. 7 and graphically in FIG. 9, an operator may understand that initialization has completed. At this stage, an operator can press or otherwise actuate actuator (310) to begin arming needle assembly (20). After actuator (310) is pressed, drive assembly (100) and/or firing assembly (140) can be activated to automatically arm or cock both piercer (22) and cutter (40). During this sequence, distal indicator (320) and intermediate indicator (322) can be switched to an off state as shown in block (434) of FIG. 7. Meanwhile, proximal indicator (324) can be switched to a flashing or blinking state as also shown in block (434) of FIG. 7. The combination of indicators (320, 322, 324) as shown schematically in block (434) of FIG. 7 and graphically in FIG. 10 can communicate that arming of biopsy device (10) is in progress.

The state of indicators (320, 322, 324) as shown schematically in block (434) of FIG. 7 and graphically in FIG. 10 can continue through the duration of the arming sequence. Once needle assembly (20) is armed as shown in block (436) of FIG. 7, indicators (320, 322, 324) can switch to the state shown schematically in block (438) of FIG. 7 and schematically in FIG. 11. In this state, proximal indicator (320) and intermediate indicator (322) are both switched to an off state. Meanwhile, distal indicator (324) is switched to a solid or continuous illumination state. In this state, indicators (320, 322, 324) communicate to an operator that the arming sequence is complete and biopsy device (10) is ready for sample acquisition.

Once indicators (320, 322, 324) are activated as shown schematically in block (438) of FIG. 7 and graphically in FIG. 11, an operator can next position biopsy device (10) for acquisition of a tissue sample. In one use, this process may include scoring tissue, injecting therapeutics into a patient, immobilizing tissue, or otherwise preparing for insertion of needle assembly (20). Once all desired preparations have been made, an operator can position the distal end of needle assembly (20) adjacent to targeted tissue of a patient. Optionally, this can also include inserting a portion of needle assembly (20) into a patient.

Once needle assembly (20) is positioned as desired, firing can be initiated by pressing actuator (310) as shown in block (440) of FIG. 7. At this stage, drive assembly (100) and/or firing assembly (140) can be activated to fire piercer (22) distally through tissue of a patient. Once actuator (310) is pressed, indicators (320, 322, 324) activate as shown schematically in block (444) of FIG. 7 and graphically in FIG. 12 for the duration of piercer (22) firing. In particular, proximal indicator (320) can be switched off, intermediate indicator (322) can be switched to a flashing or blinking state, and distal indicator (324) can be switched to an illuminated or solid state. Thus, it should be understood that the state of indicators (320, 322, 324) shown schematically in block (444) of FIG. 7 and graphically in FIG. 12 corresponds to the process of piercer (22) firing.

Once piercer (22) has fired as shown in block (446), indicators (320, 322, 324) automatically switch to the state show schematically in block (448) of FIG. 7 and graphically in FIG. 13. As can be seen, in this state, proximal indicator (320) can be switched off, while intermediate indicator (322) and distal indicator (324) can be switched to an illuminated or solid state. Thus, it should be understood that the state of indicators (320, 322, 324) shown schematically in block (448) of FIG. 7 and graphically in FIG. 13 communicates completion of piercer (22) firing to an operator.

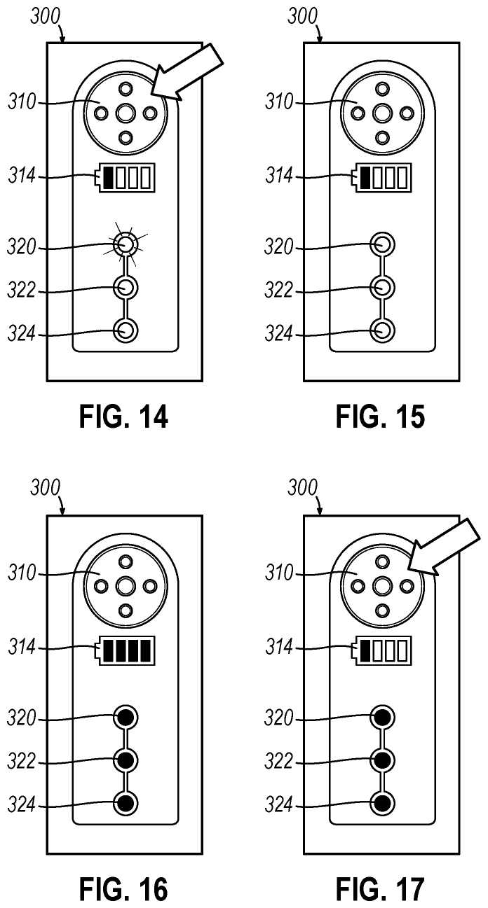

Once piercer (22) firing is complete, an operator can next begin collection of a tissue sample. To begin collection of a tissue sample, an operator can press actuator (310) as shown in block (450) of FIG. 7. Once actuator (310) is pressed, drive assembly (100) and or firing assembly (140) can be activated to fire cutter (40) distally. This movement causes distal end (42) of cutter (40) to sever a tissue sample into notch (26) of piercer. In some uses, severing of the tissue sample may be additionally facilitated by tissue naturally prolapsing into notch (26) prior to, or during, advancement of cutter (40). Simultaneously, indicators (320, 322, 324) automatically switch to the state show schematically in block (454) of FIG. 7 and graphically in FIG. 14. As can be seen, in this state, proximal indicator (320) can be switched to a flashing or blinking state, while intermediate indicator (322) and distal indicator (324) can be switched to an illuminated or solid state. Thus, it should be understood that the state of indicators (320, 322, 324) shown schematically in block (454) of FIG. 7 and graphically in FIG. 14 communicates to an operator that cutter (40) firing and the process of sample accusation is in progress. This state can continue until sample acquisition is complete.

Although the present use includes discrete actuations of actuator (310) to initiate firing of piercer (22) and cutter (40), respectively, it should be understood that in other uses, actuator (310) can be used to initiate firing of piercer (22) and cutter (40) in other ways. For instance, in one use, a single actuation or press of actuator (310) can be used to initiate firing of piercer (22) and cutter (40) in a predetermined sequence. In other words, instead of having discrete actuations of actuator (310) for piercer (22) and cutter (40), actuator (310) can be pressed once to fire piercer (22) and then cutter (40) can be subsequently fired automatically to collect a tissue sample. In other uses, a more complex actuation of actuator (310) may be used to initiate firing of piercer (22), cutter (40), or both. For instance, actuator (310) can be double tapped, triple tapped, or held for a predetermined period to initiate firing of piercer (22), cutter (40), or both. Such a more complex actuation of actuator (310) may be desirable to prevent inadvertant firing of piercer (22) and/or cutter (40). In still other uses, firing may be performed as described above, but actuator (310) can also be configured to initiate a lockout state. For instance, actuator (310) a doble tap, triple tap, or multi-second hold of actuator (310) can be configured to initiate a lockout state. In such a state, actuator (310) can be temporarily deactivated and one or more indicators (320, 322, 324) can be illuminated in a particular pattern to indicate such a lockout state. Such a lockout state may be desirable in some uses to prevent inadvertant firing of piercer (22), cutter (40), or both. In such uses, such a lockout state can be activated at any time or, alternatively, only in association with firing of piercer (22) and/or cutter (40).

After cutter (40) has fired, sample acquisition continues by activating drive assembly (100) and/or firing assembly (140) to retract piercer (22) proximally while cutter (40) remains in position. This proximal retraction of piercer (22) draws the severed tissue sample proximally to tissue sample holder (200), where the severed tissue sample can be collected by tissue sample holder (200) or any other tissue sample collection means using the process described above. For instance, shaft (242) can be rotated using keyed portion (244) to rotate wipers (250) and thereby extract the tissue sample from notch (26). Once the tissue sample is extracted from notch (26), piercer (22) can then be advanced distally to return to its original position prior to arming shown in block (430) or after initialization shown in block (420).

After sample collection is complete as shown by block (456) in FIG. 7, indicators (320, 322, 324) automatically switch to the state show schematically in block (458) of FIG. 7 and graphically in FIG. 15. As can be seen, in this state, proximal indicator (320), intermediate indicator (322), and distal indicator (324) can all be switched off. Thus, it should be understood that the state of indicators (320, 322, 324) shown schematically in block (458) of FIG. 7 and graphically in FIG. 15 communicates to an operator that cutter (40) firing and the process of sample accusation has completed.

Once sample acquisition has completed, biopsy device (10) is ready to collect additional samples as shown in block (460). At this stage, further additional samples can optionally be collected by pressing actuator (310) again and returning to block (430) of FIG. 7. In some examples, this may include an intermediate step of manipulating shaft (242) of tissue sample holder (200) to reposition any previously collected tissue samples as desired. The process described above is then repeated again starting at block (430). Alternatively, if a desired number of samples have been collected, needle assembly (20) can be removed from the patient and the biopsy procedure can be finalized. Although the present use includes actuation of actuator (310) in the sequence described above to collect additional samples, in other uses, actuator (310) can be configured to permit collection of additional samples with a hold or continuous press of actuator (310). For instance, actuator (310) can be pressed and held. In response, piercer (22) can be automatically actuated to collect additional samples until actuator (310) is released. Such a continuous press of actuator (310) may be desirable in some uses to promote rapid acquisition of additional samples. In other uses, such a feature can be omitted to avoid inadvertant acquisition of additional samples.

FIGS. 16 and 17 show certain alternative states for indicators (320, 322, 324) that can be used in addition to the states described above. For instance, FIG. 16 shows a state of indicators (320, 322, 324) that can correspond to when the probe is detached from the holster of biopsy device (10). In this state, all indicators (320, 322, 324) are set to an off state. In addition, power indicator (314) is also set to an off state. In some uses, this state may be desirable to conserve power while biopsy device (10) is not in use.

FIG. 17 shows an alternative state to the state shown in FIG. 16. In this state, probe can still be detached from holster. However, it still may nonetheless be desirable for an operator to obtain input from power indicator (314). To do so, an operator may press actuator (310) while holster is detached from probe and user interface is in the state shown in FIG. 16. Once actuator (310) is pressed, power indicator (314) can activate to communicate to an operator the level of power remaining in holster of biopsy device (10). Meanwhile, indicators (320, 322, 324) remain in the off state. After actuator (310) is pressed, power indicator (314) can remain on for a predetermined period of time. Alternatively, power indicator (314) can remain on indefinitely until either actuator (310) is pressed again or a probe is attached to holster. Alternatively, actuator (310) can be pressed in one or more predetermined sequences to activate power indicator (314). For instance, actuator (310) can be double pressed, triple pressed, or held for a predetermined period of time to activate power indicator (314). Such predetermined sequences to activate power indicator (314) may be desirable to activate power indicator (314) at any time, regardless of the state of probe and holster if power indicator (314) is otherwise deactivated.

### III. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A core needle biopsy device, comprising: (a) a needle assembly, the needle assembly including a piercer and a hollow cutter, the piercer including a sharp distal tip and a notch proximal to the distal tip, the piercer being slidably disposed within the cutter to sever a tissue sample into the notch of the piercer; (b) a drive assembly configured to selectively move the piercer and the cutter; and (c) a user interface including one or more indicators and a single button, the single button being configured to initiate a plurality of movements of the drive assembly to control arming and firing of the piercer and the cutter, the one or more indicators being configured to transition between a plurality of predetermined states to define one or more predetermined status sequences indicative of movement of the piercer and the cutter via the drive assembly.

### Example 2

The core needle biopsy device of Example 1, the drive assembly being configured to move the piercer and the cutter through an initialization sequence, an arming sequence, a firing sequence, and a sample acquisition sequence, the single button being configured to initiate the initialization sequence, the arming sequence, and the firing sequence using a single button movement associated with each of the initialization sequence, the arming sequence, and the firing sequence.

### Example 3

The core needle biopsy device of Example 2, the single button movement associated with each of the initialization sequence, the arming sequence, and the firing sequence being the same for each sequence.

### Example 4

The core needle biopsy device of Example 2, one or more of the single button movements associated with each of the initialization sequence, the arming sequence, and the firing sequence being different relative to the other single button movements for at least one of the initialization sequence, the arming sequence, or the firing sequence.

### Example 5

The core needle biopsy device of Example 2, the single button movement associated with the firing sequence being a multi-second hold of the single button.

### Example 6

The core needle biopsy device of Example 2, the single button movement associated with the firing sequence including a discrete single button movement associated with firing the piercer and firing the cutter.

### Example 7

The core needle biopsy device of Example 2, the single button movement associated either the initialization sequence, the arming sequence, and the firing sequence including a double tap of the single button.

### Example 8

The core needle biopsy device of Example 2, the single button movement associated either the initialization sequence, the arming sequence, and the firing sequence including a triple tap of the single button.

### Example 9

The core needle biopsy device of Example 1, further comprising a controller in communication with the single button and the drive assembly, the controller being configured to receive an input from the single button to drive one or more movements of the drive assembly.

### Example 10

The core needle biopsy device of Example 9, the controller being configured to receive a lockout input from the single button, the controller being configured to lock movement of the drive assembly in response to receipt of the lockout input.

### Example 11

The core needle biopsy device of Example 10, the lockout input corresponding to the single button being actuated through a predetermined lockout sequence, the predetermined lockout sequence including a long press of 2 or more seconds, a double tap, or a triple tap.

### Example 12

The core needle biopsy device of Example 9, the controller being configured to receive a multi-sample input from the single button, the controller being configured to drive a plurality of movements of the drive assembly to collect a plurality of tissue samples sequentially.

### Example 13

The core needle biopsy device of Example 12, the multi-sample input from the single button corresponding to a continuous hold of the single button.

### Example 14

The core needle biopsy device of claim 12, the multi-sample input from the single button corresponding to a continuous hold of the single button, the controller being configured to drive the plurality of movements of the drive assembly to collect a plurality of tissue samples sequentially during the duration of the continuous hold of the single button and stop the plurality of movements of the drive assembly upon release of the continuous hold of the single button.

### Example 15

A core needle biopsy device, comprising: (a) a body; (b) a needle assembly extending distally from the body, the needle assembly including a piercer and a hollow cutter, the piercer including a sharp distal tip and a notch proximal to the distal tip, the piercer being slidably disposed within the cutter to sever a tissue sample into the notch of the piercer; (c) a tissue sample holder disposed on a distal end of the body proximate a portion of the needle assembly; (d) a drive assembly positioned within the body and configured to selectively move the piercer and the cutter; and (e) a user interface including one or more indicators, a single button, and a controller in communication with the single button and the drive assembly, the single button being disposed on the body proximate the tissue sample holder and transversely centered with respect to a transverse axis of the body, the controller being configured to control one or more movements of the drive assembly using input from the single button, the one or more indicators being configured to transition between a plurality of predetermined states to define one or more predetermined status sequences indicative of movement of the piercer and the cutter via the drive assembly.

### Example 16

The core needle biopsy device of Example 15, the single button being disposed proximally of the one or more indicators.

### Example 17

The core needle biopsy device of Example 15, the controller being configured to receive a user input from only the single button.

### Example 18

The core needle biopsy device of Example 15, the controller being configured to control the drive assembly through a initialization sequence, a arming sequence, a firing sequence, and a sample acquisition sequence, the controller being configured to initiate each of the arming sequence, the firing sequence, and the sample actuation sequence in response to a user input from the single button.

### Example 19

The core needle biopsy device of Example 18, the user input from the single button being a long press, a double tap, or a triple tap.

### Example 20

A method for collecting a tissue sample using a biopsy device, the method comprising: (a) pressing a single button to initiate arming of the biopsy device, arming the biopsy device including retracting a piercer and a cutter proximally relative to a probe and holster of the biopsy device; (b) indicating the act of arming using an indicator array by adjusting a plurality of indicators of the indicator array to a predetermined first status sequence; (c) pressing the single button to initiate firing one or more of the piercer or the cutter; (d) indicating the act of firing by adjusting the plurality of indicators of the indicator array to a predetermined second status sequence different from the first status sequence; (e) acquiring a tissue sample using the piercer and the cutter; and (f) indicating the step of acquiring by adjusting the plurality of indicators of the indicator array to a predetermined third status sequence different from the first and second status sequences.

### Example 21

The method of Example 20, the step of pressing the single button to initiate firing including firing the piercer after the act of pressing the single button, the step of acquiring a tissue sample including pressing the single button to fire the cutter.

### Example 22

The method of Example 20, further comprising the step of detaching the probe from the holster; and indicating detachment of the probe from the holster by adjusting the plurality of indicators of the indicator array to a predetermined third status sequence different from the first, second, and third status sequences.

### Example 23

The method of Example 22, further comprising the step of indicating a power level associated with the holster using a power indicator by pressing the single button in a predetermined sequence, the predetermined sequence including a long press, a double tap, or a triple tap.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

We claim:

1. A core needle biopsy device, comprising:
(a) a needle assembly, the needle assembly including a piercer and a hollow cutter, the piercer including a sharp distal tip and a notch proximal to the distal tip, the piercer being slidably disposed within the cutter to sever a tissue sample into the notch of the piercer;
(b) a drive assembly configured to selectively move the piercer and the cutter; and
(c) a user interface including one or more indicators and a single button, the single button being configured to initiate a plurality of movements of the drive assembly to control arming and firing of the piercer and the cutter, the one or more indicators being configured to transition between a plurality of predetermined states to define one or more predetermined status sequences indicative of movement of the piercer and the cutter via the drive assembly.

2. The core needle biopsy device of claim 1, the drive assembly being configured to move the piercer and the cutter through an initialization sequence, an arming sequence, a firing sequence, and a sample acquisition sequence, the single button being configured to initiate the initialization sequence, the arming sequence, and the firing sequence using a single button movement associated with each of the initialization sequence, the arming sequence, and the firing sequence.

3. The core needle biopsy device of claim 2, the single button movement associated with each of the initialization sequence, the arming sequence, and the firing sequence being the same for each sequence.

4. The core needle biopsy device of claim 2, one or more of the single button movements associated with each of the initialization sequence, the arming sequence, and the firing sequence being different relative to the other single button movements for at least one of the initialization sequence, the arming sequence, or the firing sequence.

5. The core needle biopsy device of claim 2, the single button movement associated with the firing sequence being a multi-second hold of the single button.

6. The core needle biopsy device of claim 2, the single button movement associated with the firing sequence including a discrete single button movement associated with firing the piercer and firing the cutter.

7. The core needle biopsy device of claim 2, the single button movement associated either the initialization sequence, the arming sequence, and the firing sequence including a double tap of the single button.

8. The core needle biopsy device of claim 2, the single button movement associated either the initialization sequence, the arming sequence, and the firing sequence including a triple tap of the single button.

9. The core needle biopsy device of claim 1, further comprising a controller in communication with the single button and the drive assembly, the controller being configured to receive an input from the single button to drive one or more movements of the drive assembly.

10. The core needle biopsy device of claim 9, the controller being configured to receive a lockout input from the single button, the controller being configured to lock movement of the drive assembly in response to receipt of the lockout input.

11. The core needle biopsy device of claim 10, the lockout input corresponding to the single button being actuated through a predetermined lockout sequence, the predetermined lockout sequence including a long press of 2 or more seconds, a double tap, or a triple tap.

12. The core needle biopsy device of claim 9, the controller being configured to receive a multi-sample input from the single button, the controller being configured to drive a plurality of movements of the drive assembly to collect a plurality of tissue samples sequentially.

13. The core needle biopsy device of claim 12, the multi-sample input from the single button corresponding to a continuous hold of the single button.

14. The core needle biopsy device of claim 12, the multi-sample input from the single button corresponding to a continuous hold of the single button, the controller being configured to drive the plurality of movements of the drive assembly to collect a plurality of tissue samples sequentially during the duration of the continuous hold of the single button and stop the plurality of movements of the drive assembly upon release of the continuous hold of the single button.

15. A core needle biopsy device, comprising:
(a) a body;
(b) a needle assembly extending distally from the body, the needle assembly including a piercer and a hollow cutter, the piercer including a sharp distal tip and a notch proximal to the distal tip, the piercer being slidably disposed within the cutter to sever a tissue sample into the notch of the piercer;
(c) a tissue sample holder disposed on a distal end of the body proximate a portion of the needle assembly;
(d) a drive assembly positioned within the body and configured to selectively move the piercer and the cutter; and
(e) a user interface including one or more indicators, a single button, and a controller in communication with the single button and the drive assembly, the single button being disposed on the body proximate the tissue sample holder and transversely centered with respect to a transverse axis of the body, the controller being configured to control one or more movements of the drive assembly using input from the single button, the one or more indicators being configured to transition between a plurality of predetermined states to define one or more predetermined status sequences indicative of movement of the piercer and the cutter via the drive assembly.

16. The core needle biopsy device of claim 15, the single button being disposed proximally of the one or more indicators.

17. The core needle biopsy device of claim 15, the controller being configured to receive a user input from only the single button.

18. The core needle biopsy device of claim 15, the controller being configured to control the drive assembly through a initialization sequence, a arming sequence, a firing sequence, and a sample acquisition sequence, the controller being configured to initiate each of the arming sequence, the firing sequence, and the sample actuation sequence in response to a user input from the single button.

19. The core needle biopsy device of claim 18, the user input from the single button being a long press, a double tap, or a triple tap.

20. A method for collecting a tissue sample using a biopsy device, the method comprising:
(a) pressing a single button to initiate arming of the biopsy device, arming the biopsy device including retracting a piercer and a cutter proximally relative to a probe and holster of the biopsy device;
(b) indicating the act of arming using an indicator array by adjusting a plurality of indicators of the indicator array to a predetermined first status sequence;
(c) pressing the single button to initiate firing one or more of the piercer or the cutter;
(d) indicating the act of firing by adjusting the plurality of indicators of the indicator array to a predetermined second status sequence different from the first status sequence;
(e) acquiring a tissue sample using the piercer and the cutter; and
(f) indicating the step of acquiring by adjusting the plurality of indicators of the indicator array to a predetermined third status sequence different from the first and second status sequences.
